(19) United States
(12) Patent Application Publication
(10) Pub. No.: US 2019/0184058 A1
(43) Pub. Date: Jun. 20, 2019

(54) Porous Metal Devices

(71) Applicant: PorOsteon Development LLC, Menlo Park, CA (US)

(72) Inventors: Hokuto Aihara, Belmont, CA (US); John Zider, San Diego, CA (US); Robert B Zider, Portola Valley, CA (US); Gary S Fanton, Portola Valley, CA (US); Scott Carpenter, Fremont, CA (US); Thomas Duerig, Fremont, CA (US)

(21) Appl. No.: 16/315,634

(22) PCT Filed: Jul. 5, 2017
(86) PCT No.: PCT/US2017/040770

§ 371 Date: Jan. 7, 2019

(51) Int. Cl. A61L 27/06; A61L 27/56; C22C 19/03; C22C 14/00; C22C 19/00; B01D 39/20

(52) U.S. Cl. CPC A61L 27/06; A61L 27/56; C22C 19/03; A61L 2430/02; C22C 19/007; B01D 39/2034; C22C 14/00

(57) ABSTRACT

Devices comprising a component which has an open porous structure composed of an alloy of nickel and titanium or a mixture of nickel and titanium. The devices can be implanted in a mammalian body and provide desired interaction with protein, blood, ions, bone cells, and tissue. The devices are particularly useful for providing a substrate for the ingrowth of bone. In the open pore structure, preferably more than 95% of the pores having a size of 50-1000 μm, particularly 50-600 μm, with a pore size standard deviation of 250 μm or less, particularly of 150 μm or less, and an average porosity by volume of 40-80%. When the device is implanted adjacent to a cancellous bone, the porous component preferably has a modulus of 0.1-1.2 GPa. When the device is implanted adjacent to cortical bone, the porous component preferably has a modulus of 16 to 24 GPa. The devices are also useful for filtering a liquid.

### CROSS-REFERENCE TO RELATED APPLICATIONS

This application claims priority from, and the benefit of, US provisional application No. 62/358,407, filed Jul. 5, 2016, by Hokuto Aihara, John Zider, Robert B Zider, Gary S Fanton, Scott Carpenter and Thomas Duerig. The entire contents of that application are incorporated herein by reference for all purposes.

### BACKGROUND OF THE INVENTION

This invention relates to porous devices which can be implanted into a mammalian body. The devices can also be useful as filters. The invention includes the preparation and use of such devices.

### SUMMARY OF THE INVENTION

In its first aspect, this invention provides devices which can be implanted in a mammalian body and provide desired interaction with protein, blood, ions, bone cells, and tissue. In particular, the devices are useful for providing a substrate for the growth of bone. Similar devices are also useful as flow restrictors.

In its first aspect, this invention provides a device comprising a component which (1) is composed of an alloy of nickel and titanium, and (2) has an open porous structure, with more than 95%, preferably more than 98%, of the pores having a size of 50-1000 μm, preferably 50-600 μm, particularly 100-500 μm, especially 200-250 μm. the average pore size is preferably 100-600 μm. The porous structure preferably has a pore size standard deviation of 250 μm or less, particularly of 150 μm or less. In some embodiments the porous structure has an average porosity by volume of 1-90%; in other embodiments 10-90%; in other embodiments 20-90%; in other embodiments 40-80%; in other embodiments 60-80%; and preferably 40-60%.

The capillarity of the nickel-titanium component is advantageous because it promotes transportation of desired fluid materials and nutrients into the network of passageways and retention of fluid material in the structure, without the need to apply external hydraulic forces

When the component is placed adjacent to a cancellous or cortical bone in a mammalian body, the open pore structure of the component encourages bone to grow into the component. Examples of mammalian bodies are humans and animals, including dogs and horses.

The alloy of nickel and titanium comprises 30-70 atomic % titanium and 70-30 atomic % nickel, for example about 48-52 atomic % titanium and about 52-48 atomic % nickel, e.g. about 50 atomic % titanium and about 50 atomic % nickel, preferably 49 atomic % titanium and 51 atomic % nickel. The alloy consisting essentially of about 49 atomic % titanium and about 51 atomic % nickel is referred to herein as Nitinol. Where this specification describes the manufacture or modification of components composed of Nitinol, or the use of components composed of Nitinol, the description is also applicable to components composed of the other alloys of nickel and titanium described above. The alloy preferably does not, but can contain, other ingredients which do not substantially detract from the value of the porous components of the invention.

In some embodiments, the component preferably has a modulus of elasticity selected to be compatible with bone, for example 0.1-40 GPa, e.g. 0.1-24 GPa or 0.1-20 GPa, in some cases 0.1-5.0 GPa, e.g. 0.4-2.0 GPa. In some embodiments, the component has a friction coefficient of 0.1-2.0. In some embodiments, the component can withstand a tensile force of greater than 5 MPa, in other embodiments of greater than 40 MPa; in other embodiments of greater than 100 MPa. In some embodiments, the component can withstand a compression force of greater than about 1 MPa, in other embodiments of greater than 800 MPa.

The porous components of the invention can have improved impact resistance as compared to a similarly shaped component made out of a substantially solid metal or plastic. For example, the component can have an impact resistance of less one-third of the impact resistance of a similarly shaped device made out of polyetheretherketone (PEEK).

Some devices of the invention comprise first and second porous components each of which is as defined above, which are attached to each other and which differ from each other, for example in the average pore size, and/or modulus of elasticity and/or friction coefficient. Other devices of the invention comprise a first porous component as defined above and a second porous component which (a) is not a porous component as defined above, and (b) is attached to the first porous component. The second porous component can have a higher average pore size or a lower average pore size than the first component.

Some devices of the invention comprise a first porous component as defined above and a second more rigid component which increases the strength of the device and which may or may not be porous. The second component can for example be composed of a metal or a polymeric composition.

Components composed porous tantalum and porous titanium are very stiff, and as a result, provide a less than satisfactory substrate for the growth of bone. The porous nickel titanium components of the invention provide an improved substrate for the growth of bone. Furthermore, as further described below, (1) in some embodiments, the device of the invention has the ability to change shape after implantation as a result of a spontaneous change in shape when the device is heated by the warmth of the mammalian body; (2) in other embodiments, the device has sufficient elasticity to enable it to expand after it has been implanted into a cavity; for example, the outside scaffolding on the device can have a very weak, highly porous surface that can expand in a spring-like manner to accommodate any abnormalities in the geometry of the cavity.

In its second aspect, this invention provides a method of making a porous nickel-titanium device according to the first aspect of the invention.

In its third aspect, this invention provides a method of modifying a mammalian body by implanting into the body a device comprising a porous nickel-titanium component according to the first aspect of the invention.

In its fourth aspect, this invention provides a method of filtering a liquid which comprises passing the liquid through the device comprising a porous nickel-titanium component according to the first aspect of the invention.

### BRIEF DESCRIPTION OF THE DRAWINGS

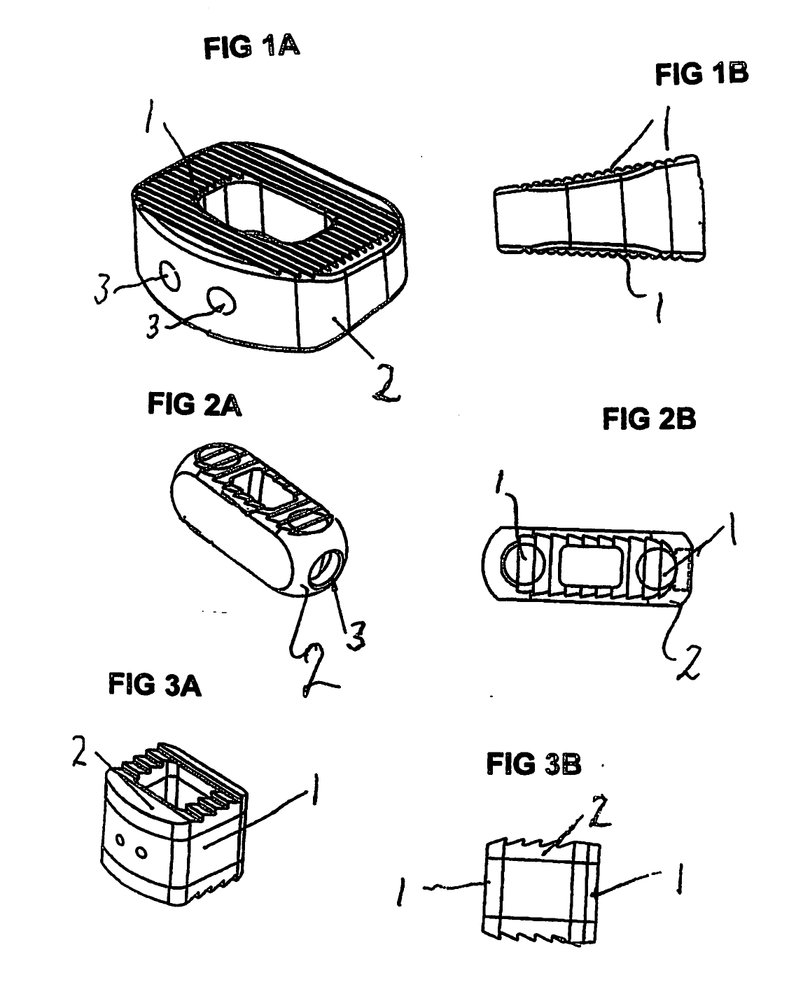

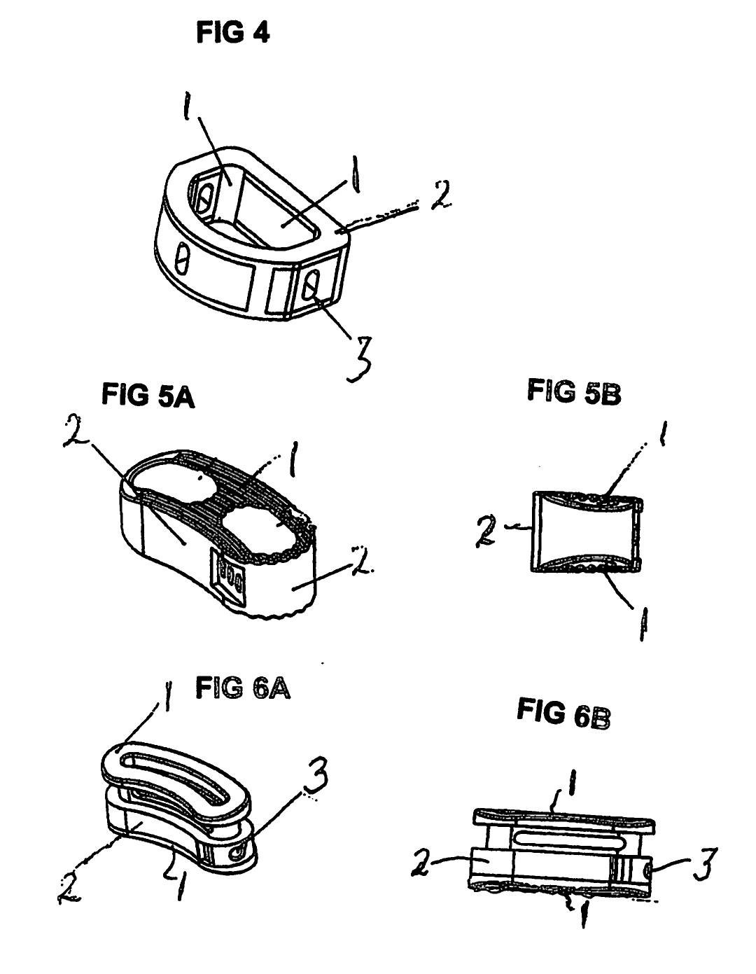

The invention is illustrated in the attached exemplary drawings, in which FIGS. 1A and 1B, FIGS. 2A and 2B, FIGS. 3A and 3B, FIGS. 5A and 5B, FIGS. 6A and 6B, FIGS. 7A and 7B, FIGS. 14A and 14B, FIGS. 17A and 17B and FIGS. 20A and 20B are, respectively, perspective and side views of various devices of the invention; and FIGS. 4, 8, 9, 12, 13, 15, 16, 18 and 19 are perspective views of other devices of the invention.

### DETAILED DESCRIPTION OF THE INVENTION

In the Summary of the Invention above, the Detailed Description of the Invention, the Examples, and the claims below, and the accompanying drawings, reference is made to particular features of the invention. These features can for example be components, ingredients, elements, devices, apparatus, systems, groups, ranges, method steps, test results and instructions, including program instructions. It is to be understood that the disclosure of the invention in this specification includes all possible combinations of such particular features. For example, where a particular feature is disclosed in the context of a particular aspect or embodiment of the invention, or a particular claim, or a particular Statement, or a particular Figure, that feature can also be used in combination with and/or in the context of other particular aspects, embodiments, claims and Figures, and in the invention generally, except where the context excludes that possibility.

The invention disclosed herein, and the claims, include embodiments not specifically described herein and can for example make use of features which are not specifically described herein, but which provide functions which are the same, equivalent or similar to, features specifically disclosed herein.

The term “comprises” and grammatical equivalents thereof are used herein to mean that, in addition to the features specifically identified, other features are optionally present. For example, a composition or device “comprising” (or “which comprises”) components A, B and C can contain only components A, B and C, or can contain not only components A, B and C but also one or more other components.

The term “consisting essentially of” and grammatical equivalents thereof is used herein to mean that, in addition to the features specifically identified, other features may be present which do not materially alter the claimed invention.

The term “at least” followed by a number is used herein to denote the start of a range beginning with that number (which may be a range having an upper limit or no upper limit, depending on the variable being defined). For example “at least 1” means 1 or more than 1, and “at least 80%” means 80% or more than 80%.

The term “at most” followed by a number is used herein to denote the end of a range ending with that number (which may be a range having 1 or 0 as its lower limit, or a range having no lower limit, depending upon the variable being defined). For example, “at most 4” means 4 or less than 4, and “at most 40%” means 40% or less than 40%. When a range is given as “ (a first number) to (a second number)” or “(a first number)—(a second number)”, this means a range whose lower limit is the first number and whose upper limit is the second number. The terms “plural”, “multiple”, “plurality” and “multiplicity” are used herein to denote two or more than two features.

Where reference is made herein to a method comprising two or more defined steps, the defined steps can be carried out in any order or simultaneously (except where the context excludes that possibility), and the method can optionally include one or more other steps which are carried out before any of the defined steps, between two of the defined steps, or after all the defined steps, except where the context excludes that possibility.

Where reference is made herein to “first” and “second” features, this is generally done for identification purposes; unless the context requires otherwise, the first and second features can be the same or different, and reference to a first feature does not mean that a second feature is necessarily present (though it may be present).

Where reference is made herein to “a” or “an” feature, this includes the possibility that there are two or more such features (except where the context excludes that possibility). Thus there may be a single such feature or a plurality of such features. Where reference is made herein to two or more features, this includes the possibility that the two or more features are replaced by a lesser number or greater number of features which provide the same function, except where the context excludes that possibility.

The numbers given herein should be construed with the latitude appropriate to their context and expression; for example, each number is subject to variation which depends on the accuracy with which it can be measured by methods conventionally used by those skilled in the art at the date of filing of this specification.

The term “and/or” is used herein to mean the presence of either or both of the two possibilities stated before and after “and/or”. The possibilities can for example be components, ingredients, elements, devices, apparatus, systems, groups, ranges and steps. For example “item A and/or item B” discloses three possibilities, namely (1) only item A is present, (2) only item B is present, and (3) both item A and item B are present.

Where this specification refers to a component “selected from the group consisting of” two or more specified sub-components, the selected component can be a single one of the specified sub-components or a mixture of two or more of the specified sub-components.

If any element in a claim of this specification is considered to be, under the provisions of 35 USC 112, an element in a claim for a combination which is expressed as a means or step for performing a specified function without the recital in the claim of structure, material, or acts in support thereof, and is, therefore, construed to cover the corresponding structure, material, or acts described in the specification and equivalents thereof, then the corresponding structure, material, or acts in question include not only the corresponding structure, material, or acts explicitly described in the specification and the equivalents of such structure, material, or acts, but also such structure, material, or acts described in the US patent documents incorporated by reference herein and the equivalents of such structure, material, or acts. Similarly, if any element (although not specifically using the term “means”) in a claim of this application is correctly construed as equivalent to the term means or step for performing a specified function without the recital in the claim of structure, material, or acts in support thereof, then the corresponding structure, material, or acts in question include not only the corresponding structure, material, or acts explicitly described in the specification and the equivalents of such structure, material, or acts, but also such structure, material, or acts described in the US patent documents incorporated by reference herein and the equivalents of such structure, material, or acts.

This specification incorporates by reference all documents referred to herein and all documents filed concurrently with this specification or filed previously in connection with this application, including but not limited to such documents which are open to public inspection with this specification.

Properties of the Porous Nickel Titanium Components.

The modulus of elasticity and other properties of the nickel-titanium component can be selected to be compatible with bone, so that the component provides a substrate into which bone can grow. In use, the devices of the invention are preferably implanted into a mammal so that a surface of the porous nickel titanium component is adjacent to cancellous or cortical bone.

In some embodiments the component has a modulus of elasticity (M) of about 0.1 GPa to about 40 GPa; in other embodiments of about 0.1 GPa to about 20 GPa; in other embodiments of about 0.1 GPa to about 5.0 GPa; in other embodiments of about 0.4 GPa to about 2.0 GPa. Cancellous bone has an anisotropic pore structure, a modulus of elasticity of 0.001-1.521 GPa and a porosity of 30-90%. Cortical bone is denser than cancellous bone and has a modulus of elasticity of 14-20 GPa and a porosity of 5-30%.

Preferably, at least the surface of the component adjacent to the cancellous or cortical bone has a modulus similar to the modulus of cancellous or cortical bone. The component preferably has a modulus which is from about 0.6 to about 1.4 times, preferably 0.8 to 1.2 times, the modulus of cancellous or cortical bone. If the component is implanted adjacent to a cancellous bone having a modulus of about 1.0 GPa, then the surface of the component adjacent to the cancellous bone preferably has a modulus of about 0.6-1.4 GPa, particularly about 0.8-1.2 GPa. If the component is implanted adjacent to a cortical bone having a modulus of about 20 GPa, then the surface of the component adjacent to the cortical bone preferably has a modulus of about 12 to 28 GPa, particularly about 16 to 24 GPa. By contrast, conventional metals such as stainless steel and titanium exhibit a modulus of elasticity of up to 210 GPa and 110 GPa respectively.

The porous nickel titanium components can exhibit dampening properties which can be helpful for joints subject to shock or impact loading and which help shield surrounding tissues from damage and promote better healing.

In some embodiments the component has a friction coefficient of about 0.1 to about 2.0. In some embodiments the porous metal device can withstand a tensile force of greater than about 5 MPa; in other embodiments greater than about 40 MPa; in other embodiments greater than 100 MPa. In some embodiments the porous metal device can withstand a compression force of greater than about 1 MPa; in other embodiments of greater than 800 MPa.

Treatments to Modify Porous Nickel Titanium Components.

The component can be subject to one or more treatments to change its properties, including but not limited to its capillarity, and/or its shape. Capillarity is indicated by the ability of the porous component to absorb a liquid (e.g. water) and/or to have a wettable surface.

Treatment of the porous component with one or more liquids, e.g. acids and other solvents, can change its capillarity, and the change can be permanent or temporary. By varying such parameters as time, temperature, and concentration of the liquid, the porosity and pore size of the component can be changed to make the component more suitable for bone ingrowth.

In some cases, chemical treatment is used to remove contaminants on the surface of the component, for example native oxides and impurities remaining after EDM or conventional machining.

Other treatments that can be used to change the characteristics of the component, e.g. to change its corrosion resistance, and/or its porosity, and/or its shape, include electropolishing, electroplating, acid etching, photo etching, microblasting, grid blasting, sandblasting, surface coating, e.g. nitriding and/or carbiding, heat treatment, plasma coating, passivation (thermal or chemical), anodizing, dip coating, sputter coating, acid etching, non-etching solvent wash, acetone (or other solvent) dip, alkali cleaning agents, milling, lathing, laser cutting, wire and sinker electro discharge machining (EDM), and additive manufacturing. An example of EDM machining includes using a nickel-titanium wire to minimize introduction of impurities to the porous metal component. A silver coating can be applied by electroplating or sputter coating.

The treatment can increase or decrease the surface energy of the component, without removal of any of the component. An increase in the surface energy increases the surface wettability and the capillarity of the porous component, while inducing a strong interaction between the surface of the component and water. This can increase cell response to the component. Other treatments can decrease the surface energy of the porous component and thus reduce its capillarity.

For example, an aggressive acid treatment increases surface roughness and surface energy. Long exposure to an acid can irreversibly remove part of the component, resulting in a smooth surface, larger pore sizes and porosity; thus affecting the capillarity of the material.

A compressive load can be applied in any way to part or all of a porous nickel titanium component, including but not limited to uniaxial press (Instron, Lloyd, bench top press), CIP (cold isostatic press)/HIP (hot isostatic press), thread rolling, cold or hot rolling/working, knurling, forming, stamping, microblasting and formation of internal or external thread by plastic deformation of a part of the component. The effect of such compressive loads is to decrease both the porosity and pore size. The application of a compressive load reduces the size of the inter-connective pores and can reduce the capillarity of the component. Thus, the overall porosity of porous nickel titanium component can be controlled by the application of an external load on the component. The application of a compressive load can be used to increase the strength of all or part of the component. The capillarity of a compressed porous nickel titanium component can in some cases be partially restored by applying chemical treatments such as those mentioned above

The porous metal component can be processed to strengthen part or all of the component. For example, a mechanical force can be applied to a portion of the device to reduce the pore size and/or compress a portion of the component. Examples of processes that can be used to apply a mechanical force include molding, stamping, CIP (Cold Isostatic Press) and HIP (Hot Isostatic Press). Reducing the pore size can strengthen a specific area of the device. Selective strengthening can also be used to shape a portion of the device. A component can be shaped to add threads or to shape a cranial implant to add the desired topography. Selective strengthening can add some plastic deformation or stress hardening to the device while still keeping an open pore structure.

The porous metal component can be processed to selectively weaken all or part of the component. For example, chemical treatment can be used to selectively remove the bulk material, subsequently increasing the porosity and pore size of a portion of the metal device. The selective strengthening and selective weakening can also be used to modify or further tune the modulus of the porous metal device.

The porous metal device can be annealed after reacting. Annealing can remove impurities, reaction by-products, and undesirable metal phases.

The surface friction characteristics of porous Nitinol can be controlled by the porosity and post machining finish. The open porosity of porous Nitinol exhibits inherent anti-migration properties. The surface friction characteristics of the porous nickel titanium component are particularly important for spinal implants, not only at the time of implantation but also for controlling subsequent migration, which is an important reason for unsatisfactory subsequent outcomes.

The surface of the porous Nitinol component preferably exhibits a high friction coefficient of at least 0.1, in particular at least 0.5, for example up to 1.2. Various machining techniques can be applied to porous Nitinol to modify the surface friction. The inherent friction can be exaggerated or suppressed by supplemental machining or other finishing operations. Machining methods include, but not limited to microblasting/grid blasting, sandblasting, nitriding/carbiding, milling, lathe, wire and sinker EDM (Electro Discharge Machining), electropolishing and acid etching. These machining techniques, and chemical processes, can be applied to control the surface finish. Rough surface finish exhibits a high friction coefficient which is beneficial for exhibiting anti-migration property. The surface finish can be controlled by applying various machining techniques. For example, EDM machining can result in a smooth surface with open pores for bone ingrowth. Some types of EDM or conventional machining can leave copper, zinc or other impurities which can be removed by a chemical treatment. To increase the friction of EDM cut parts, serrations can be made on the face or faces interfacing the bone for enhanced fixation and stability of the device. Conventional machining on a CNC Mill results in a rough surface while maintaining the majority of the pores on the surface open.

A high friction surface on the nickel-titanium component is advantageous not only because it assists at the time of implantation of the device and also because it minimizes subsequent implant migration, which can lead to a need to replace an existing implant, particularly a spinal implant.

In some embodiments of the invention, the device includes a therapeutic, biologic or bioactive material, the material preferably being on the surface of, and/or in the porous structure of, the porous nickel titanium component. The material can improve healing, and/or tissue and/or bone growth onto and into the device after it has been implanted in the patient. The material can be a timed release material.

Examples of therapeutic and bioactive agents include, but are not limited to, antibiotics, silver coating, chemotherapy drugs for the treatment of tumors, biologics, growth factors, stem cells, growth factors/BMPs (bone morphogenetic proteins)/stem cells, DBM (demineralized bone matrix)/hydroxyapatite (HA) and platelet-rich plasma (PRP), IBF, TDR, osteotomy wedges. The drugs may be associated with a biodegradable polymer for timed release.

in some devices of the invention, the porous nickel titanium component is the sole structural element. One example is a plurality of individual granules (alternatively termed “pellets” herein). The granules can be agglomerated with a biodegradable polymer and a drug into a desired shape. The granules can be introduced into a cavity in a mammalian body to promote bone growth into the cavity from an adjacent bone. The granules can be used in place of, or in addition to, other grafting materials, bone cements and devices. The granules can be introduced as a loose collection of individual granules or they can be contained in a flexible container, for example a net, which is compatible with the mammalian body and which preferably is bioabsorbable. In one embodiment, a plurality of beads of the porous nickel titanium are attached to a device, for example a relatively long and thin component, which is for example composed of a polymeric composition, e.g. a biodegradable composition. In such a device, the beads can have a central cavity through which the long and thin component passes.

The granules can for example be used in procedures such as spinal fusion, vertebral body defect (e.g. tumor), tumor replacement and sinus augmentation.

As further described below, many of the devices of the invention include, in addition to the porous nickel titanium component, an additional structural component. However, in some cases, particularly where the strength of the device is not of primary importance, the various devices described below can consist essentially of the porous nickel titanium component.

The devices of the invention optionally contain, in addition to one or more porous nickel titanium components, one or more second components which provide the device with useful properties, particularly structural properties. Some devices include at least one second component to which the porous nickel-titanium component is attached and which adds strength and/or flexibility to the device. The second component can for example be more rigid than the nickel titanium component. The second component can be solid or porous and can for example be composed of a polymeric composition, e.g. a composition based on polyetheretherketone (PEEK), or a metal, e.g. a biocompatible alloy, for example a nickel titanium alloy. The use of a suitable second component, for example a second component which is radiolucent, can have the advantage that the progress of bone growth into the device can be observed through radiographic visualization and/or antenna-enhanced MRI imaging.

Examples of devices of the invention include screws; rods; flow restrictors; dental bracket backings; dental implants; dental implant mounts; acetabular shells; acetabular augments; ankle replacements; ankle fusions; bone graft substitutes; bone/suture anchors; bone fusions; cervical, lumbar and thoracolumbar spinal fusion devices; IBF cages; cranial plates; maxillofacial plates; craniomaxillofacial(cmf) plates; cervical plates; thoracolumblar plates; devices for drug/agent delivery applications; fracture plates and rods; glenoid replacements; hip stems; interference screws; intramedullary rods; laminoplasty plugs and wedges; non-union fractures; osteochondral defects (screws and plugs); osteotomy spacers; wedge and bone fillers; patella replacements; pedicle screws; OCD screws; screws for fracture fixation; scaphoid screws; sinus augmentations; shoulder replacement; small joint arthroplasty; scaffolding for soft tissue or for tissue engineering; tendon, ligament, and tissue repair; tibial and femoral cones; tibial tray; total disc replacement; total knee replacement; toe and finger implants; tumor repair/resection; fracture rod; fixation bar for pelvis fracture or sacroiliac(SI) joint dislocation; cladding on a large bone implant; tendon repair (e.g. ACL or PCL in knee); bone or suture anchor; corpectomy; vertebral body replacement (VBR); minimally invasive spine (MIS) devices; total disc replacement (TDR) endplate coatings, expandable cages; and intermedullary implants for SI joint fusion.

In one embodiment, the device of the invention has a first shape before it is implanted into a mammal, for example an elastically deformable shape, and a second shape after it has been implanted, for example as the result of elastic recovery of the porous metal component and/or another component. In one embodiment, the change in shape takes place spontaneously in response to a change in temperature after the device has been implanted. For example, the device can be at a first temperature and have a first shape at the time it is implanted into a mammal, and change spontaneously to a second shape after implantation when heated or cooled to body temperature. The change in shape can be produced by a change in shape of a porous metal component and/or by a change in shape of another component of the device. Preferably the change in shape results from a component composed of a nickel-titanium alloy.

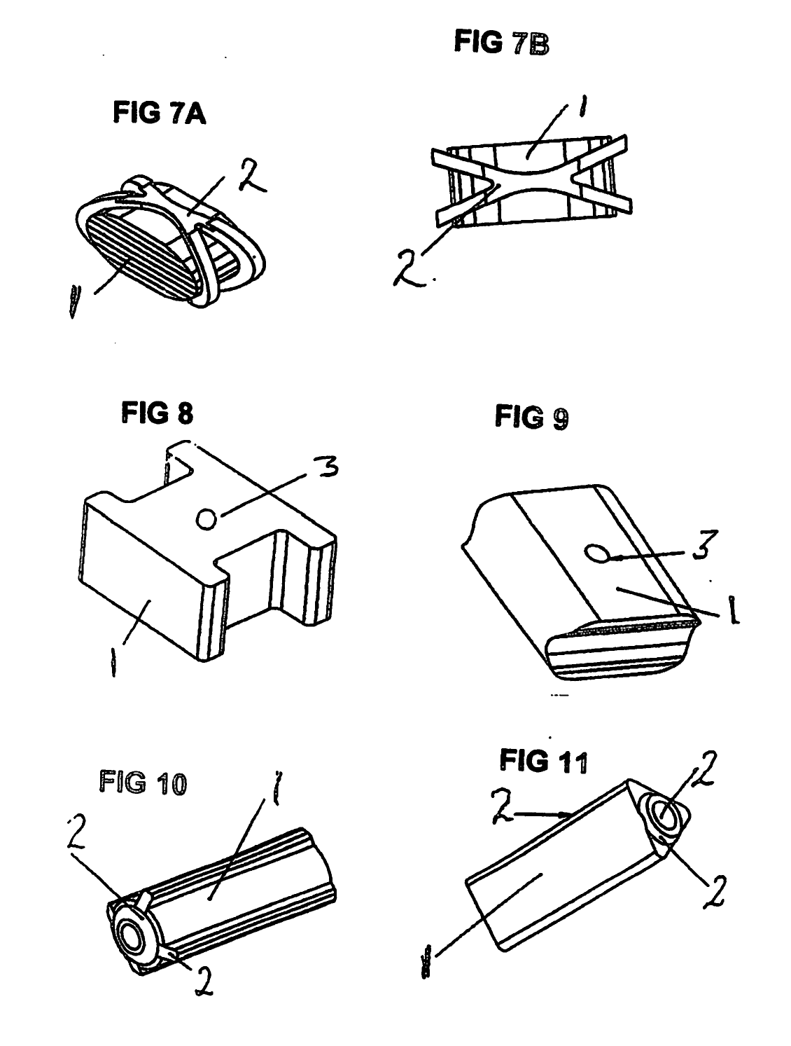

In one embodiment, the device comprises two components, e.g. components providing outer parts of the device, whose relative positions can be changed, manually and/or with the aid of a third component. For example, the height and/or another dimension of a device can be changed so that the device can contain porous nickel titanium components of different dimensions. Examples of such devices are shown in FIG. 6A and 6B and in FIGS. 7A and 7B.

The devices disclosed herein can comprise a first exposed surface having a first surface characteristic, e.g. coefficient of friction and/or wicking capability, and a second exposed surface having a second surface characteristic. One or both of the first and second exposed surfaces can be composed of the porous metal.

Further information about Devices of the Invention.

The device preferably exhibits stiffness similar to that of the cancellous or cortical bone to which it is adjacent, and also provides migration resistance and shock resistance. By making use of a second component which is composed of PEEK or a similar polymeric composition, it is possible to solve the problem of obtaining bone ingrowth and device fixation, combined with radiopacity for bone ingrowth examination. A device including a second component which is composed of PEEK or similar polymeric composition can be constructed in several ways, including, but not limited to mechanical attachment, reflow of PEEK into the porous Nitinol by applying a combination of heat and pressure, adhesive bond, insert molding, press fitting and ultrasonic welding.

One type of composite device comprises one or more outer surfaces which, when the device is implanted, interface the bone and are màde from porous Nitinol, and one or more other components made of non-metallic or metallic material. Bone ingrowth can be achieved up to the interface between the porous Nitinol and the other component. The attachment of the dissimilar materials can be achieved in any way, including, but not limited to, compression molding, diffusion bonding, laser welding, and mechanical attachment.

Potential benefits include but are not limited to: radiographic visualization of fusion, particularly inside central cavity space of cage devices; bone ingrowth beyond the implant-to-bone interface in all devices; and long-term implant stability particularly in comparison with surface-enhanced bone “on-growth” technologies (e.g., plasma spray, sand blasted surface).

is An interbody fusion (IBF) device can comprise a first component composed of a metallic or non-metallic material, preferably PEEK, with one or more porous Nitinol components, e.g. posts, attached to the first component. The mode of the attachment includes but it is not limited to press fit, threading and compression molding. The porous Nitinol component can extend through the height of the device and facilitate bone growth. The porous Nitinol component(s) can not only participate in the fusion process, but also replace the need for any marking devices with radiolucent cage material as seen as tantalum marker on PEEK IBF devices. The interconnective pore space can be used to load therapeutic agents or biologics for time release drug delivery or further promoting bone growth.

The device can include a smooth casing, optionally a skeletal casing, around a porous nickel-titanium component, for example a cage or block. The casing can be made from any suitable material, including, but not limited to, polymeric materials (including biodegradable/bioresorbable polymers) and metallic materials. The casing can be made of a biodegradable material and/or the porous Nitinol material can be loaded with therapeutic agents or biologics for time release drug delivery for further promoting bone growth. The casing can protect any surrounding tissue, organs, and vital structure during the placement of the devices, and/or provide implant structure integrity, and/or facilitate placement of the device, e.g. laterally prior to “flipping” the device into final position. The casing can for example be capped, mechanically attached, plasma-sprayed, diffusion bonded, or compression molded. Selected regions of the implant can be also compression molded and/or attached with a smooth device rather than a full casing surrounding the implant.

In one embodiment, the device includes an expandable cage made of metallic or non-metallic material with porous Nitinol coated endplates. The attachment of porous Nitinol endplates can be achieved in any way, e.g. by diffusion bonding, compression molding, mechanical attachment, or laser welding.

In some embodiments, for example a standalone IBF compression molded device, the device includes holes for placement of screws in lumbar, cervical or thoracic interbody fusion. Freestanding holes can be made of metallic or non-metallic material. Mechanical attachment of pre-threaded or non-threaded “caps” can be applied to reduce chance of particles flaking when using a fully porous Nitinol cage.

The porous Nitinol material can be attached to other components, e.g. a metallic or non-metallic cage, in any way, e.g. compression molding, diffusion bond, or mechanical attachment. The cage material can also be porous Nitinol wherein holes for the screws can be created as a pilot hole. Initial fixing of the device can be achieved by an external screw penetrating the upper and lower endplate of an IBF device.

Selective strengthening of the interface between the screw and porous Nitinol is achieved when the screw is threaded through the pilot hole, improving the stability of device. Other benefits includes reduction of costs associated with instrumentation, OR time, and patient complications.

Some embodiments of the invention, e.g. an IBF device, comprise a plurality of composed of porous Nitinol sheets stacked on top of each another for MIS. The attachment between the Nitinol sheets can be achieved by mechanical connection.

In some embodiments, a fully porous Nitinol component is used for fusing a damaged lamina. The pores in the Nitinol can be used to load therapeutic agents or biologics for time release drug delivery or further promoting bone growth.

In some embodiments, the device is a SI joint fusion device having a porous Nitinol outer surface material interfacing the bone and a solid or partially porous inner core with similar or dissimilar core. The preferred inner core material is wrought Nitinol to accommodate flexibility. The porous outer surface of porous Nitinol facilitate boney growth, while the inner core provides strength. The connection between the porous Nitinol outer material and the inner core can be achieved in any way, for example by compression molding, diffusion bonding, press fitting, self-tapping, or threading. The interconnective pore space on porous Nitinol can be used to load therapeutic agents or biologics for time release drug delivery or further promoting bone growth.

In one embodiment of the invention, a first surface of the device is provided by a porous nickel titanium component of the invention to enable bone ingrowth or ligament attachment and a second surface, preferably an opposing surface, is provided by a component which composed of a different material and which has a smooth surface that can be placed against soft tissue, adjacent bone, vital organs, and/or articulating surfaces. The two components can be attached to each other in any way, for example by compression molding, diffusion bonding, or mechanical attachment. Devices of this type can be used for example for TDR, endplate, patella, glenoid, tibial and femoral condyle, ankle fusion, toes/finger joints, ligament repair, and craniomaxillofacial application.

In another embodiment, the device is a screw which is made completely or partially from porous Nitinol. The porous Nitinol may be placed along the interfacial surface of the device. Porous Nitinol may provide part or all of the interfacial surface of the screw. A non-metallic or metallic rod may be placed in the central cavity of the porous Nitinol material for added strength. Porous Nitinol may be placed in the middle segment in a form of sleeve, threaded or unthreaded; of the screw for promoting bone ingrowth. Porous Nitinol strip(s) with or without threads may be placed along the distal direction of the device for promoting ingrowth and implant stability. The connection between the porous Nitinol and the remainder of the device maybe achieved in any way, for example by compression molding, diffusion bonding, laser welding, and mechanical attachment. Threads on porous Nitinol may be created in any way, for example by thread rolling, EDM, or conventional machining, and may produce selective strengthening of the interfacial surface of porous Nitinol. Screw devices of this type can be used as pedicle screws and, for example, in dental, ACL, MCL, PCL reconstruction.

In one procedure, a dental implant comprises a donut shaped base made from porous Nitinol is initially press fitted or screwed in the region of interest. The outer or inner surface of the base may be threaded or unthreaded. A dental implant made from titanium, a titanium alloy, wrought Nitinol or other biocompatible material, preferably a titanium alloy, is composed of two thread diameters. The distal thread is screwed into the inner hole of the base, while the proximal threads interface bone or surrounding tissue. The outer diameter of the base is smaller than the diameter of the proximal thread, allowing implant fixation. The porous Nitinol base facilitates bone ingrowth, which allows greater fixation of the implant to the interfacing bone.

In another embodiment, the device is a standalone or conjunctional bone rod composed of the nickel titanium alloy. The outer surface of the rod can be harder than the interior of the rod and can be prepared by hardening the outer surface, for example by roll threading or by a die. The device can be biomechanically similar to diaphysis of the long bone. Preferably, the internal porous Nitinol matrix exhibits properties similar to cancellous bone, while the hardened surface material exhibits properties similar to cortical bone. Hardening the surface interfacing the bone retains open pores for bone ingrowth. Potential applications include but not limited to: small and large bone repair/fixation, trauma, upper and lower extremities (e.g., fingers, toes, ankle fusion device, pedicle screws, and rods.)

In a similar device, the hardened porous Nitinol surrounds a central component composed of a non-metal or metal, for example Nitinol. The attachment between the two components can be achieved in any way, for example by press fitting, compression molding, diffusion bonding, laser welding, and mechanical attachment. The central component rod accommodates the natural bending encountered during daily activities.

In another embodiment, the device comprises a central rod component and two end components which comprise porous nickel titanium components of the invention. The end components can be coated onto the outer surfaces of the rod or secured to the ends of the rod. The device can example be used for fusing bone and/or stabilizing and implant without the need for additional hardware fixation. Applications for such device include for example SI joint fusion, fingers/toes and pedicle screws.

In another embodiment of the invention, a porous nickel titanium device of the invention is custom fabricated into a sheet for cranial, maxillofacial or sacral reconstruction, using die or mold specific for each patient, or for ligament or tissue repair. The thickness of the porous Nitinol mesh can be as thin as 0.5 mm.

Different shapes of the porous nickel titanium component of the invention can be made by machining a block of the material to a wedge, block, cylindrical, or custom shape by EDM, conventional machining, or additive manufacturing. Applications includes but not limited to: osteotomies, tumor replacement, laminoplasty. osteochondral defects (OCD), tibial, femoral cones, acetabular augments, hammertoes, craniomaxillofacial defects such as eye socket repair, sinus augmentation.

In another embodiment, the device is a two-way plug. Such a device is useful, for example, for the surgical correction of hammer toe. One of the methods for treating hammer toe is fusing the joint together to prevent the toe from bending in an abnormal direction. The plug may be composed entirely or partially from porous Nitinol. The attachment of the porous Nitinol piece(s) on the plug may be achieved by threading, bonding, diffusion bonding, sintering, compression moldings or mechanical attachment. The attachment of the joint may be achieved by inserting a two way plug (dual conical shape), between the two junctions. The plug may have serrations, threads, or other surface features to station the device in the region of interest. The junction is secured by staples, bracket, or other conventional methods, and/or used as a pin for non-load bearing fusion of fractured bones. Applications include but not limited to: small bone repair/fixation-finger and toes.

Some devices of the invention comprise a nickel-titanium component, which may be the porous nickel titanium component of the invention, which has a first shape at a storage temperature and a second shape at a second, higher temperature. The nickel-titanium component can for example have a transformation temperature (Af) between −65° C. and 50° C., preferably between −65° C. and 0° C. The change in shape of the device can take place before the device is implanted, but preferably takes place after the device has been implanted and has been heated to body temperature.

In one embodiment, the device is an acetabular shell device made partially or completely from porous Nitinol material matching the modulus of cancellous bone to reduce stress-shielding. The shell can either be a hemi-spherical block device or individualized sections of porous Nitinol, making up a hemi-spherical structure. The device includes components of a different material. The attachment between the different components may be achieved for example by compression molding, diffusion bonding, laser welding and mechanical attachment.

In one embodiment, the device is a bracket unit for orthodontic application. The bracket can be composed of completely or partially from porous Nitinol. Porous Nitinol provides an interface between the orthodontic bracket and the tooth. A second component which is not composed of porous Nitinol can be connected to one or more porous Nitinol components. The bracket and the Nitinol components can be connected in any way, e.g. by adhesion bonding, mechanical attachment, diffusion bonding, compression molding, UV curing, laser welding, sintering, or other addition manufacturing methods, (e.g., electron beam melting(EBM), direct melt laser sintering(DMLS), selective laser sintering(SLS) or selective laser melting(SLM)). Using such technologies as EBM or direct laser melt sintering, the bracket piece can be grown using porous Nitinol thin film as a base.

The malleable property of porous Nitinol conforms to the curvature of each tooth. The interconnected pore structure exhibits large surface and the excellent wettability property of the material absorbs the adhesive to create a stronger bond between the bracket to the tooth.

In some embodiments, the porous nickel titanium component is in the form of a shapeable sheet that can be used for cranial, maxillofacial or sacral reconstruction. Porous Nitinol exhibits malleability which is beneficial for closely matching the anatomical shape for patient-specific cranial, maxillofacial, or sacral reconstruction while providing mechanical support which is amenable for bone ingrowth. Geometry can be extracted from the patient's CT scan or other means and porous Nitinol sheet can be shaped or molded to match the anatomy of the patient for the region of interest. Sheets of porous Nitinol can be produced by various machining methods, including but not limited to conventional machining, milling and EDM. They can also be produced in a desired final shape.

When the porous Nitinol sheet is plastically deformed and shaped into the desired region for reconstruction, the region where the plastic deformation took place exhibits greater stiffness. The bulk porous material, as well as the area where the deformation was induced, exhibit selective strengthening while maintaining open porosity for fluid transfer to aid in rapid bone ingrowth to stabilize the implant. Shaping of the porous Nitinol can be achieved including, but not limited to uniaxial pressure, isostatic pressing, and shaping using a die.

In another embodiment, the device is a flow restrictor which can be used in filtration, particle capture, flow control, wicking and gas/liquid contacting applications. The average pore size can be controlled which affects the resistivity of the flow. Cleaning detergent or any substance that dissolve in aqueous solution used in the case of water treatment can be infused in the porous matrix for shorten or prolonged time release. The flow restrictor can also have machined holes.

Preparation of Devices of the Invention.

One method of preparing the porous nickel-titanium components of the invention is Combustion Synthesis(CS) or Self-Propagating High-Temperature Synthesis(SHS). In such methods, two or more elemental powders (in this case, a mixture comprising nickel powder and titanium powder) are reacted with one another to form a more stable compound, thereby releasing heat sufficient to self-propagate the reaction. Preferably, the amount of heat released from the reaction of the two powders is sufficient to bring the temperature of the mixture close to the melting point of the new compound that is formed. The method can be initiated by placing the compacted powder mixture in a furnace.

In some embodiments, one or more additional metal powders are added to the compacted powder mixture to increase or decrease the (Af) transformation temperature of the product. Examples of such optionally added metal powders include one or more of nanocrystalline NiTi, tantalum, niobium, magnesium, cobalt, chromium, iron and molybdenum.

In some embodiments a filler material is included in the compacted powder mixture. The filler material can have a known size and shape and can controllably change the pore size in the resulting porous metal device. The filler material can decompose during the reaction resulting in a porous metal device with increased pore sizes. Examples of such optional fillers include sodium chloride, ammonium hydrogen carbonate, and urea.

In other embodiments, no filler is used in the compacted powder mixture or reaction process and the compacted powder mixture consists essentially of nickel powder and titanium powder.

In some embodiments, a dense or rigid component is included in the compacted powder mixture prior to the reaction. The dense or rigid component can include a solid metal or a porous metal. The metal can include a biocompatible alloy. One example of a metal is a nickel-titanium alloy. The dense or rigid component can include a solid or porous metal or polymeric composition, for example a polymeric composition comprising a polyetheretherketone (PEEK)

A multiple layer sequential ignition process can be used. For example, a small device can be made with a first porosity and modulus, then surrounded and packed with a different powder recipe followed by ignition. The process can be repeated for one or more additional layers, with each layer ignited on top of the other to produce layers having different modulus values or other properties.

Combustion Synthesis(CS) or Self-Propagating High-Temperature Synthesis(SHS) can be used to produce porous nickel-titanium which is suitable for bone graft from both cancellous bone and cortical bone.

The porous Nitinol can for example have an average porosity of 10-90%, an average pore size of 100-600 μm, and modulus of elasticity 0.1-40.0 GPa. The desired porosity of the porous Nitinol can be achieved between 1-90% can be tailored to the desired application by varying the apparent density. The porosity is preferably at 40-80%, in order to maintain appropriate material and mechanical properties as a material for bone replacement. The porous material has highly connective porosity and can be made with average pore size of 50-600 μm, e.g. 100-600 μm, preferably 50-400 μm, which aids in blood absorption, transfer nutrients, and facilitates bone apposition and eventual bone ingrowth.

In some embodiments, the compacted powder mixture is formed inside a mold having an internal volume with a desired shape for the porous metal component. In that case, the product has a desired shape corresponding to the internal volume of the mold.

Sintering is another method of preparing devices having a desired shape. For example, devices such as acetabular shell, augments, or tibial cones may be fabricated by sintering into a final shape or near final shape. Either Nitinol powder or elemental powders of Nickel and Titanium may be used to perform sintering. The latter is a “Hybrid SHS” process wherein the SHS takes place while performing sintering for the creation of Nitinol intermetallic in the form of the final shape. Variables including but not limited, to time, temperature, and pressure can be altered to optimize the process. Conventional sintering can also be performed on elemental or compound granules or powder mixtures. The powder mixture can consist of Nitinol powder. Phase transformation temperature can be specified and controlled on the Nitinol powder to incorporate shape memory or superelasticity property in the final product.

The material of the mold must withstand the high reaction temperature of the sintering or combustion synthesis process. The material of choice for the mold includes, but is not limited to stainless steel, quartz, and ceramic.

Combustion Synthesis and Sintering with Spacers.

Porous Nitinol produced by combustion synthesis or sintering of carefully selected powders provides a method of forming intermetallic compounds that exhibit high porosity, often greater than 50%. The conventional method of controlling the porosity and pore size of the synthesized product can be categorized to powder variation and process conditions.

An alternative method of controlling and creating pores in porous Nitinol is to include spacers in the Nickel and Titanium powder or Nitinol powder mixture during the mixing step. Examples of spaces are sodium chloride, ammonium hydrogen carbonate and urea.

The porosity and pore sizes can be altered by the amount and size of the spacers. In some cases, the spacers burn off during the high exothermic reaction of combustion synthesis, or during a sintering process, so that no residue is left in the finished product. In other cases, the spacer is dissolved after synthesis by solution treatment, sintering, or chemically.

Methods of Bonding Elemental Nickel and Titanium Powder to Metallic Substrates by Combustion Synthesis

In one embodiment, a porous Nitinol article is attached to an underlying partially or fully porous substrate.

Combustion synthesis of carefully selected powders provides a method of forming intermetallic compounds that produces highly porous product, often with porosities greater than 50%. While such porous structures are of great utility for certain medical reasons, such as bony ingrowth and matching the modulus of bone, they are not satisfactory for all purposes because their strength and toughness is low compared to commonly used orthopedic materials, such as cobalt-chrome alloys and wrought titanium.

Some devices of the invention comprise a component composed of porous nickel-titanium and a substrate of high strength material, so that the device combines the strength and toughness of the substrate, and the characteristics of porous material. The wrought material may contain threads, knurling, or machined patterns on the surface to enhance the attachment strength between the porous material and the substrate. A layer of porous material can also be attached to a porous substrate with different material and mechanical properties than the surround layer.

In devices having a metallic substrate, the substrate can be any partially or fully dense metal, but is preferably composed of titanium or a nickel-titanium alloy or other material with providing adequate mechanical properties. If the substrate takes part in the combustion synthesis of the nickel-titanium alloy, it must have a melting point sufficiently high to prevent it from melting during the combustion process.

Some devices of the invention comprise a nickel-titanium porous layer adjacent to a partially sintered substrate or another porous substrate with a different porosity. The nickel-titanium porous layer typically has a porosity over 50% and can have porosity either higher or lower than the core substrate. Preferably both the nickel-titanium porous layer and the porous substrate have highly-connective porosity and have average pore sizes between 100-600 μm to assist in blood absorption and facilitate osseointegration into the device.

This device can be constructed in several ways, including, but not limited to mechanical attachment, reflow of the PEEK into the porous Nitinol by applying a combination of heat and pressure, adhesive bond, insert molding, press fitting and ultrasonic welding.

The product of the synthesis can undergo further processing, for example as described above, to change its shape and/or surface characteristics and/or porosity.

The devices of the invention can optionally be made by a process which includes one or more of the following steps (i) to (vii).
(i) A preformed polymeric component is attached to a porous nickel-titanium component by heating one or both of the components and pressing them together; (ii) a preformed polymeric component is attached to a porous nickel-titanium component by radio frequency or ultrasonic bonding; (iii) a metal sheet is attached to a porous nickel-titanium component by heating the metal sheet to a temperature near its melting point, and pressing it against the porous nickel-titanium component; (iv) a mold is filled with a metal powder which is to become the porous nickel-titanium component, and the powder ignited; (v) at least a part of the exposed surface of the porous nickel-titanium component is subjected to electrical discharge machining; (vi) the exposed surface of the nickel-titanium component is subject to differential machining, differential blasting or differential electrical discharge machining, thus producing a nickel-titanium component whose exposed surface comprises a first area having a first surface characteristic, e.g. coefficient of friction and/or wicking capability, and a second area having a second surface characteristic; (vi) the exposed surface of the porous nickel-titanium component is subject to different additive manufacturing methods including but not limited, to electron beam melting and selective laser sintering; and (vii) a desired shape is grown on the surface of porous nickel-titanium component using similar or dissimilar material.

The devices of the invention can comprise a first component composed of the nickel-titanium porous material and a second component which is attached to the first component in any way which leaves at least a part of the first component exposed and available for bone ingrowth.

The second component can be composed of a polymeric composition, e.g. one consisting of or comprising a polyetherether ketone. The second component can for example be attached to the first component by molding, press fitting or compression molding. The second component, the first component, or both the first and second components can be heated in any way, including radio frequency or ultrasonic heating.

In devices of the invention containing a first component composed of porous nickel-titanium and a rigid second component, the connection between the first and second components can for example make use of mechanical connection such as screws, and nuts and bolts.

Additive manufacturing methods can be implemented to print a second component onto a first component composed of the porous nickel-titanium material; and vice versa, a first component composed of a porous nickel-titanium material can be printed on top of the second component, for example a metallic or polymeric base substrate.

When the second component is a thin film metal sheet, the sheet can be heated close to its melting point and then press fitted to the first component for a mechanical bond to the porous metal. One or both of the first and second components can then be attached to a substrate such as Ti, CoCr, or other material. The interface metal film provides a bigger footprint for the attachment as well as bonding compatibility with the substrate, such as porous Nitinol or a titanium film to a titanium implant. Alternatively, the metal can be melted and then sprayed or poured onto the porous metal. Other similar or dissimilar material can be deposited or grown using electron beam melting (EBM) or selective laser sintering (SLS). In each case, the layer of metal can be attached to an implant.

Some devices of the invention can have some elasticity so that the device can be implanted into a slightly larger cavity in the mammal in order to minimize any mismatch or voids, similar to a self-expanding stent. In other devices, an outside scaffolding on the porous metal device can be used with a very weak, highly porous surface that can expand in a spring like manner to accommodate any abnormalities in the geometry of the cavity. In other devices the properties of the device are selected so that it has “heat to recover” properties between a low temperature, e.g. room temperature, and body temperature, thus allowing the device, after it has been implanted into a cavity, to expand into any voids in the cavity.

The devices disclosed herein can result in enhanced imaging through use of an antenna. For example, bone ingrowth can be observed post-surgery by using antenna enhanced MRI imaging.

The invention is illustrated by the following Examples.

### EXAMPLE 1

A cylindrical specimen of porous Nitinol with dimensions of 0.250″±0.05″ OD×1.5″ in length (6±1.3 mm OD×38 mm in length) was fabricated by EDM (Electro Discharge Machining). The specimen had an average porosity of 64.3% and an average pore size of 216±57 μm. The porous Nitinol had a modulus of elasticity (GPa) of 1.56, an ultimate tensile strength (MPa) of 27, a porosity (%) of 64.3, a pore size (μm) of 216, and pore size standard deviation (μm) of 57.

An external thread was formed by thread rolling over a length of 0.5″ (12.7 mm) at one end of the specimen. The local deformation of the specimen along the surface of the thread resulted in an increase in toughness, a reduction of the pore size to about 100 μm (i.e. still within the preferred pore size of 50-400 μm ), and a reduction of the porosity to about 50%. The remaining 1″(25.4 mm) of the specimen was unchanged.

### EXAMPLE 2

A porous Nitinol cylindrical specimen with dimensions 20 mm OD×38 mm in length was prepared. The specimen had an average porosity of 64.3% with an average pore size of 216±57 μm. The porous Nitinol had a modulus of elasticity (GPa) of 1.56, an ultimate tensile strength (MPa) of 27, a porosity (%) of 64.3, a pore size (μm) of 216, and pore size standard deviation (μm) of 57, having an average porosity of 64.3%

An internal thread was created by first creating a pilot hole with an OD of between 2.6-3.7 mm and 13 mm in depth. Two different types of screws were drilled into the pilot hole to approximately 12.7 mm in depth. The insertion of the screw produced local deformation on the inner surface of the porous Nitinol, which created a localized deformation of the thread pattern on the inner lining of the internal thread. A pull out test was performed to characterize the shear strength of the two types of screw in the porous Nitinol. The results are shown in the tables below.

Pull out strength of wood screw

| D major | D minor | Length | Pitch | Surface Area |
|---|---|---|---|---|
| (mm) | (mm) | (mm) | (#) | (mm2) |
| 3.88 | 2.27 | 12.7 | 9 | 76 |

| Specimen | Load | Pilot Hole | Shear Strength | Shear Strength |
|---|---|---|---|---|
| # | (N) | (in) | (MPa) | (Psi) |
| 1 | 4596 | 0.106 | 60.5 | 8771 |
| 2 | 4060 | 0.097 | 53.4 | 7748 |
| 3 | 4259 | 0.104 | 56.0 | 8128 |
| Average | 4305 | 0.102 | 56.6 | 8216 |

Pull out strength of steel screw

| D major | D minor | Length | Pitch | Surface Area |
|---|---|---|---|---|
| (mm) | (mm) | (mm) | (#) | (mm2) |
| 4.1 | 3.03 | 12.7 | 20 | 81.9 |

| Specimen | Load | Pilot Hole | Shear Strength | Shear Strength |
|---|---|---|---|---|
| # | (N) | (in) | (MPa) | (Psi) |
| 1 | 2413 | 0.144 | 31.8 | 4612 |
| 2 | 3963 | 0.138 | 52.1 | 7556 |
| 3 | 3135 | 0.142 | 41.3 | 5991 |
| Average | 3170 | 0.141 | 41.7 | 6053 |

The average shear strength between the porous Nitinol specimen and two (2) screws tested were 56.6 MPa or 8.216 PSI for the wood screw and 41.7 MPa or 6.053 PSI for the machine screw. The inner surface of porous Nitinol underwent plastic deformation, which selectively strengthened the material. The inner surface of the porous Nitinol was able to withstand a high load prior to yielding to the pull force on the thread.

### EXAMPLE 3

Impact tests were performed on three (3) specimens of porous Nitinol with 1.0″ (25.4 mm) OD with a thickness of 0.25″ (6.35 mm) and were compared with porous titanium and PEEK material with the same dimensions and sample size.

The impact force for each specimen was recorded. A lower impact force reading indicated that the force induced by a hammer on each specimen was absorbed by the material prior to being transmitted to the transducer; while a high impact force reading indicated that little or no force was absorbed by the material. Hence, material for bone replacement should exhibit lower impact reading because the external impact force is absorbed by the material minimizing the residual force transmitted to the surrounding bone. The results are shown in the table below. The porous Nitinol exhibited on average 36% and 29% less than PEEK and porous titanium specimen, respectively. The impact resistance of the device minimized the transmission of external force to the receding end of the force through the device.

Impact force of porous Nitinol, Porous Titanium, and PEEK material.

| | Impact G Force (G) | | |
|---|---|---|---|
| | Porous | Porous | |
| Specimen | Nitinol | Titanium | PEEK |
| 1 | 412.9 | 579.4 | 658.5 |
| 2 | 429.1 | 642.6 | 707.9 |
| 3 | 441.2 | 586.7 | 646.1 |
| Average | 427.7 | 602.9 | 670.8 |
| Std. Dev. | 82.4 | 32.5 | 31.4 |
| % Delta to | | 29% | 36% |
| Porous Nitinol | | | |

### EXAMPLE 4

### Capillarity Study

A study was performed to measure the capillarity characteristics of porous Nitinol. The average porosity was 64%. The open porosity was determined to be 95.2% of 64%. The relative percentage of open porosity was determined by saturating the porous samples in DI water and weighing the total absorbed water. The porous Nitinol was obtained by powder metallurgy method by means of a self-propagating high-temperature synthesis or combustion synthesis with annealing afterward. Each specimen was machined by EDM. Each specimen had a standard cylindrical shape (010.0±0.25 mm×30.0±0.10 mm long).

Each specimen was suspended in air, with 2-4mm of the test specimen submerged in a water reservoir having a predetermined weight. The reservoir was placed on top of a scale and the weight of the reservoir was measured every 0.5 seconds. 3 trials were performed for each specimen. The weight of the water was converted to volume, and the total volume of water wicked by each specimen was calculated. The results were averaged. The average percent of open volume wicked was about 25% after 0.5 seconds, about 39% after 1.0 seconds, about 47% after 1.5 seconds, about 55% after 2 seconds, about 60% after 2.5 seconds, about 63% after 3 seconds, about 69% after 3.5 seconds, about 75% after 4 seconds, about 77% after 4.5 seconds, about 79% after 5 seconds, about 81% after 5.5 seconds, about 83% after six seconds about 87% after 6.5 seconds, about 88% after 7 seconds, about 90% after 7.5 seconds, about 93% after 8 seconds about 86% after 8.5 seconds, about 98% after 9 seconds, about 99% after 9.5 seconds and 100% thereafter.

### EXAMPLE 5

Porous Nitinol having an average porosity of 68.7% was used to provide an outer layer on a core substrate composed of porous Nitinol having an average porosity of 64.3%. The outer layer had a modulus of elasticity (GPa) of 0.93, and ultimate tensile strength (MPa) of 15.1, a pore size (μm) of 456 and a pore size standard deviation(μm) of 109. The core substrate had a modulus of elasticity (GPa) of 1.56, an ultimate tensile strength (MPa) of 27, a pore size (μm) of 216 and a pore size standard deviation(μm) of 57.

The resulting product had a porosity between 40 to 80% for both the layer and the core. Similar products can be prepared with one or both of the layer or the core having higher or lower average porosity or pore sizes. The interconnectivity of the pores promotes the ingrowth of biological tissues and facilitates fluid transfer.

### EXAMPLE 6

Porous Nitinol having an average porosity of 68.7% was used to encapsulate a solid Nitinol tube. The porous nitinol had a modulus of elasticity (GPa) of 0.93, an ultimate tensile strength (MPa) of 15.1, a pore size (μm) of 456 and a pore size standard deviation (μm) of 109.

### EXAMPLE 7

Samples A-C are porous Nitinol components produced by the SHS process. The porosities for samples A-C are 63±1%, 63±1%, and 68±1%, respectively. The is average pore sizes for samples A-C are 211, 213, and 203 μm, respectively. The tables below report the pore size distributions, the pore characteristics and the mechanical properties of the samples.

| | Sample A | Sample B | Sample C |
|---|---|---|---|
| % with pore size | | | |
| 0-50 μm | 0 | 0 | 1 |
| 51-100 μm | 9 | 8 | 11 |
| 101-200 μm | 46 | 45 | 47 |
| 201-300 μm | 28 | 30 | 27 |
| 301-400 μm | 12 | 11 | 10 |
| 401-500 μm | 3 | 4 | 2 |
| >501 μm | 2 | 2 | 1 |
| Pore size(μm) | | | |
| <50 | 0 | 0 | 1 |
| 50-500 | 98 | 98 | 98 |
| >500 | 2 | 2 | 1 |
| Pore dimensions(μm) | | | |
| Minimum | 41 | 38 | 8 |
| Maximum | 780 | 811 | 649 |
| Mean | 211 | 213 | 203 |
| Standard deviation | 102 | 106 | 101 |
| Mechanical Properties | | | |
| Young's Modulus (GPa) | 1.13 | 1.12 | 1.21 |
| Ultimate compression | >710 | >710 | >716 |
| strength (MPa) | | | |
| Strain at maximum | >72.8 | >70.7 | >70.1 |
| stress (%) | | | |

The Drawings.

In the accompanying drawings:—

Reference numeral 1 denotes a porous nickel-titanium component of the invention. The component can be loaded with one or more therapeutic agents or biologics as described above.

Reference numeral 2 denotes a component which is composed of a material which can be inserted into a mammalian body, for example, a metal, e.g. titanium or tantalum, or a polymeric composition, for example a polymeric composition based on PEEK, for example a polymeric composition which is radiolucent and thus permits radiographic visualization of fusion inside a central cavity or around the outer surface of a device. The component 2 can alternatively be composed of (i) a porous nickel-titanium component of the invention, which may be the same as or different from the component denoted by reference numeral 1, (ii) a porous nickel-titanium component which is not a porous nickel-titanium component of the invention, (iii) a component which is not composed of a nickel titanium alloy and which is optionally porous.

Reference numeral 3 denotes a hole or a recess which can be used to assist in manipulating the device or through which a screw can be inserted to secure the device in place.

Reference numeral 5 denotes a window through which the interior of the device can be viewed.

In some cases, one or more of the exterior surfaces of the device is ridged or otherwise serrated to assist its retention on a desired surface of the mammalian body.

FIGS. 1A and 1B show a minimally invasive spinal (MIS) device with major surfaces which are composed of porous nickel-titanium and are formed with ridges to promote friction.

FIGS. 2A and 2B show an interbody fusion (IBF) device in which the nickel-titanium components are posts that are fitted into the remainder of the device by press fitting, threading or compression molding. The remainder of the device is made of a metallic or non-metallic material, preferably PEEK, and defines a cavity into which bone can grow. This material not only participates in the fusion process, but also replaces the need for any marking devices with radiolucent cage material as seen as tantalum marker on PEEK IBF devices. The ends of the device are identical, so that the device can be inserted through either end, and, if need be, subsequently “flipped”.

FIGS. 3A and 3B show a device of the invention. The upper and lower surfaces of the device are composed of porous Nitinol and are serrated.

FIG. 4 shows a device having a smooth skeletal casing around a porous nickel-titanium cage.

FIGS. 5A and 5B show a device having a central core containing cavities and composed of PEEK, and upper and lower surfaces composed of porous Nitinol.

FIGS. 6A and 6B show a device comprising a pair of telescoping units 2 which enable the height of the device to be changed, and having upper and lower surfaces composed of porous Nitinol.

FIGS. 7A and 7B show a device which comprises a number of nickel-titanium sheets of the invention, and whose height can be changed by changing the number of the sheets.

FIGS. 8 and 9 show laminoplasty devices which are useful, for example, for fusing a damaged lamina. The devices may consist of the nickel-titanium component of the invention. Some or all of the edges of the device can be rounded to minimize damage to surrounding tissue or interfacing bone.

FIGS. 10 and 11 show sacroiliac (SI) joint fusion devices comprising one or more nickel-titanium components of the invention surrounding or partially surrounding a core of another material that provides strength. The inner core can for example be made of wrought Nitinol to accommodate flexibility.

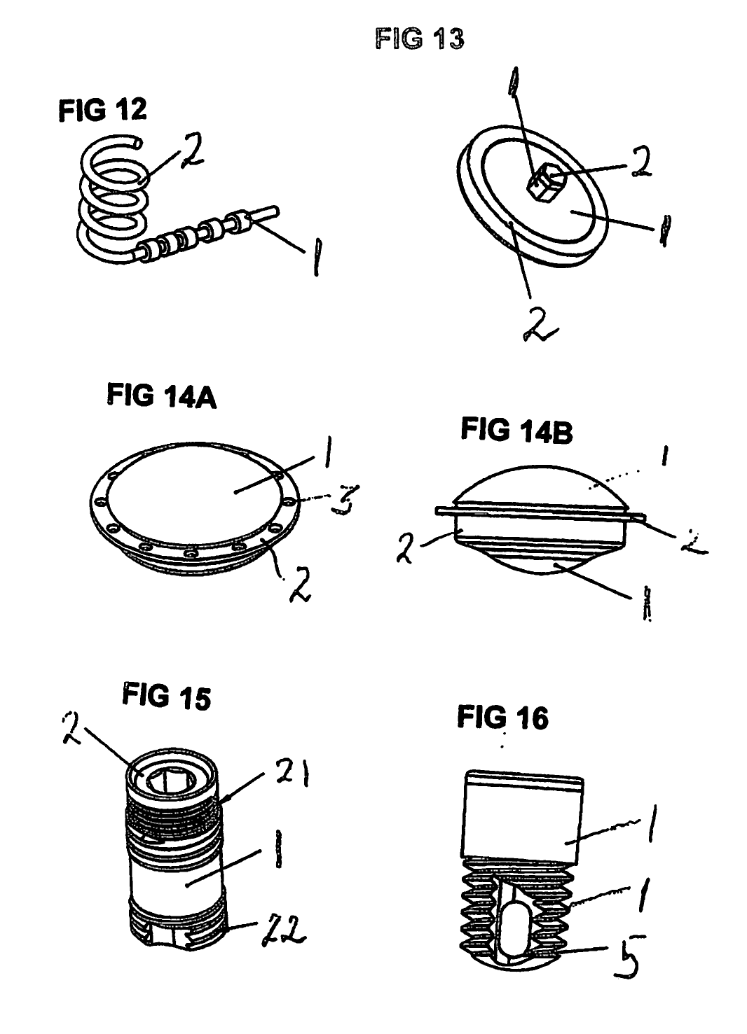

FIG. 12 shows a device which comprises a spiral component, composed for example of a polyamide or other polymeric composition, which has one end supporting a plurality of porous nickel-titanium components of the invention. The spiral component can be made of a biodegradable material. The device can be placed in a region of the vertebral height restoration.

FIG. 13 shows a primary patella device. The parts of the device which are not composed of porous nickel-titanium can for example be made of ultra high molecular weight polyethylene.

FIGS. 14A and 14B show another patella device. The device comprises a suture ring which is sandwiched between a porous nickel-titanium component and a base of a suitable polymer, for example ultra high molecular weight polyethylene. The suture ring is for example composed of titanium or a titanium alloy.

FIG. 15 is a screw device which comprises (1) a core of a suitable high-strength material, for example titanium or a titanium alloy and (2) a thread surrounding the core which is composed of porous nickel-titanium. The core has an internal shape that can be used to turn the screw.

FIG. 16 is a screw device which is similar to the device in FIG. 15, but which includes a window 5 through which observation can be made.

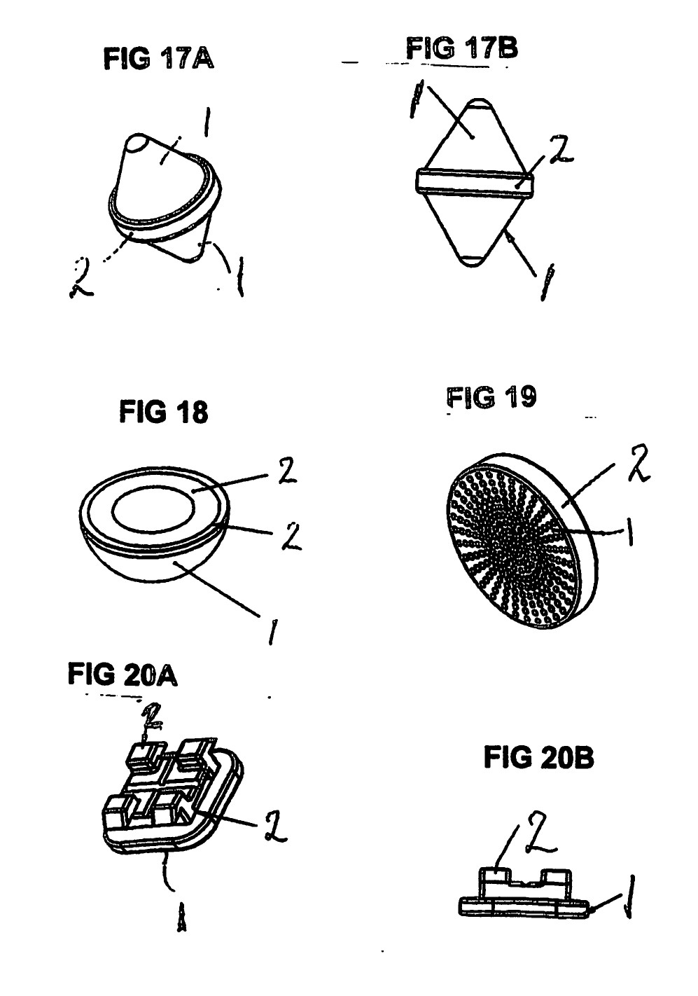

FIGS. 17A and 17B show a wedge-shaped bone filler. The material between the two porous nickel-titanium components can be porous or nonporous.

FIG. 18 shows an acetabular shell device. The flat inner surface of the device can for example be composed of a metallic, polymeric or ceramic material.

FIG. 19 shows a porous nickel-titanium component of the invention in the form of a flow restrictor. In the manufacture of the component, the average pore size can be controlled to provide a desired resistance to the flow of a liquid.

FIGS. 20A and 20B show a dental bracket. The base of the bracket is a porous nickel-titanium sheet of the invention and can be attached to a previously prepared unit composed of a different material. Alternatively, the remainder of the bracket can be fabricated by addition manufacturing (for example EBM or DMLS

Statements.

The following statements describe and define particular embodiments of the invention.

Statement 1. Porous metal devices and methods for manufacturing porous metal devices comprising nickel and titanium are disclosed herein.

Statement 2. Statement 1 wherein the porous metal device formed in the manufacturing step has a modulus of elasticity of about 0.1 to about 40.GPa.

Statement 3. Statement 1 or 2 wherein the manufactured porous metal device includes one or more of: an average pore size of about 100 μm to about 600 μm, a pore size standard deviation of about 250 μm or less, an average porosity for the porous device of about 40% to about 80%, and greater than about 95% of the pores having a size of about 50 μm to about 1000 μm.

Statement 4. Any of Statements 1-3 wherein the mixed nickel and titanium source includes 45-55 atomic % titanium and 45-55 atomic % nickel.

Statement 5. Statement 4 wherein the mixed nickel and titanium source includes about 50 atomic % titanium and about 50 atomic % nickel.

Statement 6. Any of statements 1-5 wherein forming the compacted powder mixture comprises applying pressure to the mixed nickel and titanium source to create a packing density in the compacted powder mixture of about 1.29 g/cm³ to about 6.39 g/cm³.

Statement 7. Any of statements 1-6 wherein reacting includes igniting or heating the compacted powder mixture to initiate a combustion synthesis or self-propagating high temperature synthesis reaction.

Statement 8. Statement 7 wherein reacting includes a reaction temperature within the compacted powder mixture of less than a melting point of the mixed nickel and titanium source.

Statement 9. Any of statements 1-8 which includes a step of processing the porous metal device to achieve a desired shape.

Statement 10. Statement 9 wherein processing the porous metal device to achieve the desired shape includes one or more of the following methods: microblasting, grid blasting, sandblasting, milling, lathing, laser cutting, wire and sinker electro discharge machining (EDM), electropolishing, and acid etching.

Statement 11. Any of statements 1-10 wherein the porous metal device one of: a screw for fracture fixation, an interference screw, an osteotomy spacer, a scaphoid screw, a cranial and maxillofacial plate, a fracture rod, a fixation bar for pelvis fracture or Sacroiliac (SI) joint dislocation, a dental implant and implant mount, a cervical and lumbar IBF implants, as cladding on a large bone implant, a plugs for osteochondral defects, an OCD screw, a pedicle screws, a bone or suture anchor, as soft tissue scaffolding or for tissue engineering, for tendon repair, or as a bone graft substitute.

Statement 12. Any of statements 1-11 wherein the manufacture includes placing the compacted powder mixture in a furnace.

Statement 13. Any of statements 1-12 further comprising inserting a dense component in the compacted powder mixture prior to reacting.

Statement 14. Statement 13 wherein the dense component comprises solid metal or solid plastic.

Statement 15. Statement 13 wherein the dense component comprises porous metal or porous plastic.

Statement 16. Any of statements 1-15 which includes electropolishing, acid etching or photo etching, the porous metal device to increase the pore size of the porous metal device and/or to modify a surface characteristic of the porous metal device.

Statement 17. Any of statements 1-16 further comprising treating the porous metal device to improve a corrosion resistance of the metal device.

Statement 18. Any of statements 1-17 further comprising treating the porous metal device by one or more of electropolishing, electroplating, acid etching, nitriding, carbiding, plasma coating, anodizing, dip coating, and sputter coating.

Statement 19. Any of statements 1-18 further comprising annealing the porous metal device.

Statement 20. Any of statements 1-19 further comprising selectively strengthening a portion of the porous metal device.

Statement 21. Any of statements 1-20 further comprising selectively weakening a portion of the porous metal device.

Statement 22. Any of statements 1-21 further comprising mechanically or chemically treating the porous device to modify the pore distribution of the device.

Statement 23. Statement 22 wherein the porous device is treated to modify the pore distribution to be about 60% to about 80%.

Statement 24. Any of statements 1-23 further comprising forming a second porous material comprising nickel and titanium over a portion of the porous metal device.

Statement 25. Statement 24 wherein the second porous material has a different pore structure than the porous metal device.

Statement 26. Statement 24 wherein the second porous material has a larger average pore size than the porous metal device.

Statement 27. Statement 25 wherein the second porous material has a smaller average pore size than the porous metal device.

Statement 28. Any of statements 1-27 comprising providing a filler material in the compacted powder mixture with the nickel and titanium source.

Statement 29. Any of statements 1-27 wherein the compacted powder mixture consists essentially of nickel powder and titanium powder.

Statement 30. Any of statements 1-27 wherein no filler is used in the compacted powder mixture.

Statement 31. Any of statements 1-28 wherein the treatment improves the capillarity of the porous metal device.

Statement 32. Any of statements 1-31 loading a therapeutic agent in the porous metal device.

Statement 33. Statement 32 wherein the therapeutic agent comprises one or more of bone growth factor, silver coating, and antibiotics.

Statement 34. Any of statements 1-33 wherein the compacted powder mixture is formed inside a mold having an internal volume with a desired shape for the porous metal device.

Statement 35. Statement 34 comprises forming the porous metal device with the desired shape of the internal volume of the mold.

Statement 36. A metal device comprising:

a porous mixture of nickel and titanium having an open pore structure, the pore structure including a pore distribution of greater than about 95% of the pores having a size of about 50 μm to about 1000 μm.

Statement 37. Statement 36 wherein greater than about 98% of the pores have a size of about 50 μm to about 600 μm.

Statement 38. Statement 35 or 36 wherein the pore distribution includes an average pore size of about 100 μm to about 600 μm.

Statement 39. Any of statements 36-38 wherein the pore distribution includes a pore size standard deviation of about 250 μm or less.

Statement 40. Any of statements 36-39 wherein the pore structure includes an average porosity of about 40% to about 80%.

Statement 41. Any of statements 36-40 wherein the metal device has a modulus of elasticity of about 0.1 GPa to about 40 GPa.

Statement 42. Statement 41 wherein the metal device has a modulus of elasticity of about 01 GPa to about 24 GPa.

Statement 43. Statement 41 wherein the metal device has a modulus of elasticity of about 0.1 to 5.0 GPa.

Statement 44. Statement 41 wherein the metal device has a modulus of elasticity of about 0.4 to 2.0 GPa.

Statement 45. Any of statements 36-44 wherein the metal device has a friction coefficient of about 0.1 to about 2.0.

Statement 46. Any of statements 36-45 further comprising a second porous material comprising a mixture of nickel and titanium formed over a portion of the porous mixture of nickel and titanium, the second porous material having a different pore structure than the porous mixture of nickel and titanium.

Statement 47. Statement 46 wherein the second porous material has a higher average pore size than the porous mixture of nickel and titanium.

Statement 48. Any of statements 36-47 further comprising a rigid component, wherein the porous mixture of nickel and titanium is formed over a portion of the rigid component.

Statement 49. Statement 48 wherein the rigid component comprises plastic or metal.

Statement 50. Any of statements 36-49 wherein the metal device is adapted for implantation within a mammalian body.

Statement 51. Any of statements 36-50 wherein the metal device is a screw or rod.

Statement 52. Any of statements 36-50 wherein the metal device is configured as one of: a screw for fracture fixation, an interference screw, an osteotomy spacer, a scaphoid screw, a cranial and maxillofacial plate, a fracture rod, a fixation bar for pelvis fracture or Sacroiliac (SI) joint dislocation, a dental implant and implant mount, a cervical and lumbar IBF implants, as cladding on a large bone implant, a plugs for osteochondral defects, an OCD screw, a pedicle screws, a bone or suture anchor, as soft tissue scaffolding or for tissue engineering, for tendon repair, or as a bone graft substitute.

Statement 53. Any of statements 50-52 further comprising a therapeutic agent within a portion of the open pore structure of the porous metal device.

Statement 54. Statement 53 wherein the therapeutic agent comprises one or more of: bone growth factor, silver coating, or antibiotic agent.

Statement 55. Any of statements 36-54 wherein the metal device includes 45-55 atomic % titanium and 45-55 atomic % nickel.

Statement 56. Any of statements 36-54 wherein the metal device includes about 50 atomic % titanium and about 50 atomic % nickel.

Statement 57. Any of statements 36-56 wherein the metal device has an improved impact resistance relative to a similarly shaped device made out of a substantially solid metal or plastic.

Statement 58. Statement 57 wherein the metal device has an impact resistance of less than half of an impact resistance for a similarly shaped device made out of polyetheretherketone (PEEK).

1. A device comprising a component which
(1) is composed of an alloy of nickel and titanium which comprises 30-70 atomic % titanium and 70-30 atomic % nickel, and
(2) has an open porous structure, with more than 95% of the pores having a size of 50-1000 μm.

2. A device according to claim 1 wherein the alloy comprises 48-52 atomic % titanium and 52-48 atomic % nickel.

3. A device according to claim 2 wherein the average pore size is 100-600 μm, the pore size standard deviation is 250 μm or less and the average porosity by volume is 40-80%.

4. A device according to claim 2 wherein the component has the modulus of elasticity of 0.1-40 GPa.

5. A device according to claim 2 which can withstand a tensile force of greater than 40 MPa.

6. A device according to claim 2 which comprises, in addition to the component defined in claim 1, a second component which (i) is not porous and (i) is composed of a metal or a polymeric composition which is based on PEEK.

7. A device according to claim 2 which comprises, in addition to the component defined in claim 1, a second component which (i) is not porous, (ii) is composed of a polymeric composition, and (iii) has a first shape at a temperature below body temperature and spontaneously changes to a second shape when heated to body temperature.

8. A device according to claim 7 wherein the component as defined in claim 1 has a first shape at a temperature below body temperature and spontaneously changes to a second shape when heated to body temperature.

9. A method of preparing a device according to claim 1 which comprises reacting a mixture comprising nickel powder and titanium powder by Combustion Synthesis (CS) or Self-Propagating High-Temperature Synthesis (SHS).

10. A method according to claim 9 wherein the mixture includes one or more of nanocrystalline NiTi, tantalum, niobium, magnesium, cobalt, chromium, iron and molybdenum.

11. A method according to claim 9 wherein the mixture comprises one or more of sodium chloride, ammonium hydrogen carbonate or urea.

12. A method of modifying a mammalian body which comprises implanting into the body a device comprising a porous nickel-titanium component as claimed in claim 1.

13. A method according to claim 12 wherein the porous nickel-titanium component is implanted adjacent to a cancellous bone and has a modulus of 0.1-1.2 GPa.

14. A method according to claim 12 wherein the porous nickel-titanium component is implanted adjacent to a cortical bone and has a modulus of 16 to 24 GPa.

15. Method of filtering a liquid which comprises passing the liquid through the porous nickel-titanium component of a device as defined in claim 1.
